(19) United States
(12) Patent Application Publication
(10) Pub. No.: US 2004/0259073 A1
(43) Pub. Date: Dec. 23, 2004

(54) Charge perturbation signature methods and devices for membrane analysis

(76) Inventors: Ariang Hassibi, Palo Alto, CA (US); Siavash Ghazvini, Menlo Park, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 10/794,131

(22) Filed: Mar. 5, 2004

Related U.S. Application Data
(60) Provisional application No. 60/452,645, filed on Mar. 7, 2003.

(51) Int. Cl. C12Q 1/00; G01N 33/53

(52) U.S. Cl. 435/4; 205/777.5

(57) ABSTRACT

The methods and apparatus disclosed herein concern the detection of potential differences across membrane structures. Particular embodiments of the invention concern methods and apparatus for detection of potential differences across membrane structures and measuring their responses in terms of specific ion absorption and desorption, following a variety of external perturbations, such as voltage gradients, triggering reagents, ionic fluxes, heat shocks, and mechanical vibrations. Such responses may be measured using charge perturbation signature (CPS) methods and apparatus. In specific embodiments, the methods may be used as a form of patch clamp technique allowing for highly multiplexed assays with applications in cell research and drug discovery.

### BACKGROUND

This is a non provisional application of provisional application No. 60/452,645, filed Mar. 7, 2003, and claims priority thereof.

1. Field

In various embodiments, the present invention relates to the field of detection of potential differences across membrane structures. Particular embodiments concern methods and apparatus for detection of potential differences across membrane structures and measuring their responses in terms of specific ion absorption and desorption, following a variety of external perturbations, such as voltage gradients, triggering reagents, ionic fluxes, heat shocks, and mechanical vibrations. Such responses may be measured using charge perturbation signature (CPS) methods and apparatus. In specific embodiments, the methods may be used as a form of patch clamp technique allowing for highly multiplexed assays with applications in cell research and drug discovery.

2. Background

Mass transfer processes, involving the movement of particles from one location of an electrolyte to another location, generally arise from differences in electrical potential (drift) or concentration (diffusion) at the two locations or from movement of a volume element of electrolyte (convection). Typically, when an ion-selective discontinuity in an electrolyte medium exists, for example in the form of a membrane, mass transfer processes drive the system toward an equilibrium state. However, the resulting state does not necessarily exhibit a homogeneous concentration of every permeable ionic species. This may be related, for example, to the presence of non-permeable ions that can cause a non-homogeneous distribution of counter-ions. In such a system, if either the permeability characteristics of the membrane or the composition of the electrolyte is changed, a temporary state with transient ionic fluxes will occur. Transient ion flux continues until the system reaches a new equilibrium state. In this transient phase ionic fluxes will result from diffusion and drift. Such ionic fluxes may potentially be detected using various electrochemical methods and devices.

Living cells create an inner microenvironment by surrounding themselves with a lipid membrane. These lipid bilayer membranes are practically impermeable to most of charged particles inside and outside the cell. This allows the maintenance of concentration gradients which are required for cell function. Specialized integral membrane transport proteins carry ions across the membrane and regulate the concentration gradients across the membrane. Cell transporter activity underlies a variety of physiological functions, including electrical excitability of cells, neurohormone or neurotransmitter release, transport of substances across membranes and various signaling processes in cells.

The mechanisms by which cells transport ions and small molecules across membranes can generally be categorized as either facilitated diffusion or active transport. In facilitated diffusion, transmembrane proteins create a water-filled pore or channel through the membrane. Certain ions and small hydrophilic molecules may pass through the pore by diffusion. Typically, there is some type of gating mechanism that provides selectivity or specificity for which molecules may pass, as well as controlling when the channels are open. The channels can be opened or closed according to the needs of the cell. In various cells and membranes, the channels may be ligand-gated, voltage-gated, light-gated or mechanically-gated.

In active transport mechanisms, transmembrane proteins known as transporters use the chemical energy of ATP to transport ions or small molecules across the membrane against their concentration gradient. Active transport may be used, for example, to maintain concentration gradients of sodium, potassium and calcium ions across the cell membrane.

Measurement of transient ion fluxes and/or transport processes are of use for a variety of applications in cell biology, pharmaceutical discovery or characterization, medical research, etc. Presently, ion flux measurement is typically performed using the patch-clamp technique developed by Sakmann and Neher (e.g., Neher et al., Pflugers Arch. 375:219-228, 1978; Sackmann, Fed. Proc. 37:2654-59, 1978; Hamill et al., Pflugers Arch. 391:85-100, 1981). That technique permits the direct measurement of currents through ion transporters with high time resolution. It is the only technique that is presently capable of accurately detecting conformational changes of single ion transporter proteins in real time. The method has been used to assess the action of pharmaceutical agents or other regulatory molecules directly on target proteins.

Although the patch-clamp method and its variations have been used to investigate the function of a variety of transport proteins or lipid bilayer configurations, the technique requires a superior ionic “seal” between the two sides of the membrane for accurate measurements. Such seals naturally exist in cells with intact cell membranes or other types of lipid vesicle structures. However, it becomes problematic when examining membrane fragments or isolated and reconstituted transporter proteins, for example in synthetic lipid bilayer membranes.

Current methods of membrane analysis in an array or high throughput format have two major problems. One is the challenge of adequately sealing the membrane in vitro to isolate electrolytes on either side of the membrane and eliminate ion leakage. The second impediment is the limited number and/or structure of the transmembrane proteins that can be placed in a membrane and tested using patch-clamp techniques. A need exists for a rapid, accurate method of measuring ion fluxes or transport processes, that can be used with array or high throughput formats.

### DESCRIPTION OF THE DRAWINGS

FIG. 1 describes a diffusion potential created by the gating of the ionic channels in a vesicle with a lipid bilayer outer membrane.

FIG. 2 describes a diffusion potential created by the gating of the ionic channels in a vesicle with lipid bilayer outer membrane and its detection via an electrode system.

FIG. 3 describes a typical charge perturbation signature system (CPS) analyzing the diffusion potential of ion influx into vesicles, triggered by specific reagents.

### DESCRIPTION OF ILLUSTRATIVE EMBODIMENTS

The present invention addresses a long-felt need in the art by providing methods and apparatus for measuring the diffusion potential of ions absorbed or released by ion selective membranes or lipid vesicles (e.g., cells) using charge perturbation signature. In various embodiments, the methods and apparatus do not require the isolation of membrane components containing specific transporters, but may be applied to intact cells, organelles or other membrane-enclosed compartments.

Terms that are not otherwise defined herein are used in accordance with their plain and ordinary meaning. As used herein, “a” or “an” may mean one or more than one of an item.

Detection of Ion Fluxes by Charge Perturbation Signature

Methods and apparatus of general use in charge perturbation signature (CPS) analysis are disclosed in more detail in U.S. patent application Ser. No. 10/040,303 by Pourmand and Hassibi, filed on Oct. 19, 2001, the entire text of which is incorporated herein by reference.

Ion Flux

In general, vesicles enclosed by ion-selective membranes (e.g., mammalian cells) may contain a number of different ion-selective transporters exhibiting different ionic selectivity and gating characteristics. As discussed above, ion flux may result from facilitated diffusion, which is driven by the electrochemical potential for a given transport species, or active transport, where flux may occur against an electrochemical gradient for the transported species. In general when a transport channel is activated for a specific ion (e.g. K⁺ or Ca²⁺), a temporal (transient) diffusion potential is created outside the membrane. The transient diffusion potential is a function of ionic flux, diffusion coefficient, and the concentration of other charged species in the solution surrounding the vesicle. The transient diffusion potential can be used to measure the ion flux rate across the membrane enclosing the cell or vesicle.

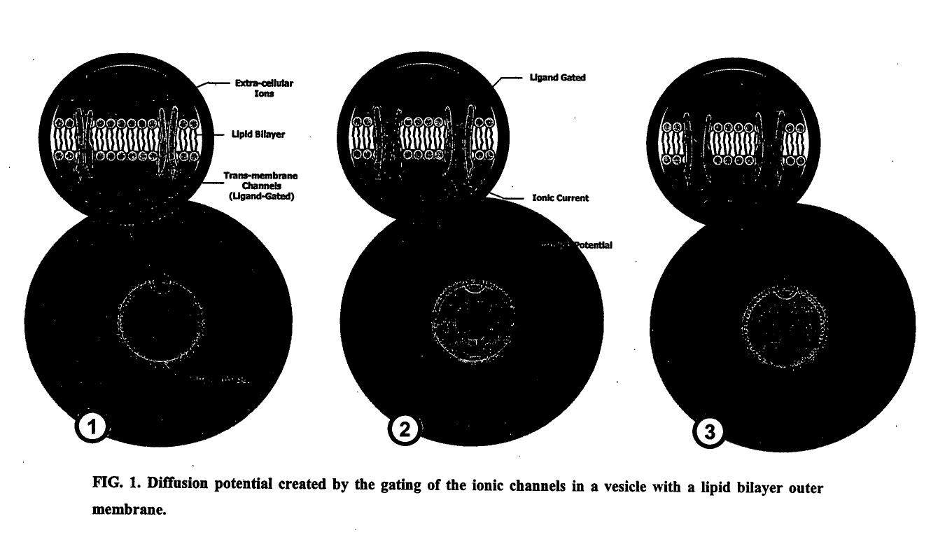

FIG. 1 illustrates a membrane-enclosed vesicle containing a ligand-gated transmembrane channel. In this case, exposure of the vesicle to the ligand, which may be a neurotransmitter, cytokine, pharmaceutical agent or other cell regulatory compound will open the gate, allowing ion flux to occur. A temporal (transient) diffusion potential is created in the surrounding medium. Over a short period of time, the diffusion potential will dissipate due to closing of the gated channels or equilibration of the transported ion across the membrane and diffusion of counterions through the medium.

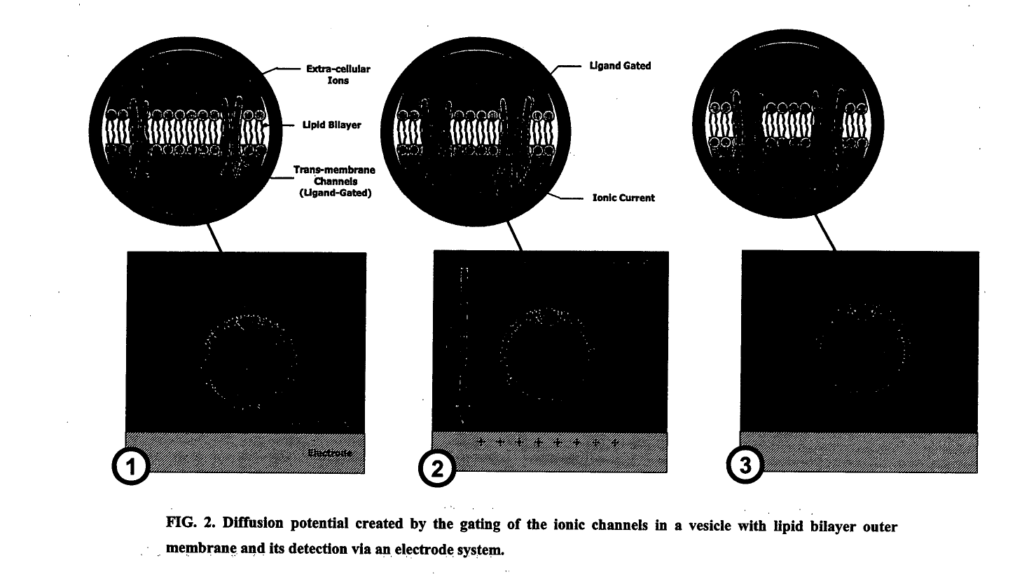

The diffusion potential from absorption or desorption of specific ionic species may be detected in the non-equilibrium state by measuring the voltage difference between two or more different locations in the solution where transient ion flux is present. As shown in FIG. 2, if an electrode is placed close enough to the target vesicle for the diffusion potential profile to be significant, the electrode can sense the temporal ionic flux as a transient voltage potential difference between the electrode and the bulk solution. Typically, the electrode must be located within a couple of diffusion lengths from the point of flux (i.e., the membrane bound vesicle). The transient voltage potential signal is a function of the distance from the electrode to the membrane, the ionic strength of the solution, the number of open channels and the rate of transport through each channel. Consequently analysis of the acquired signal can be used to determine the characteristic of the ionic flux.

Charge Perturbation Signature Method

An exemplary apparatus and system that may be used in the practice of charge perturbation signature (CPS) analysis was disclosed in Provisional U.S. patent application Ser. No. 60/440,670 by Arjang Hassibi, filed Oct. 19, 2002, the entire text of which is incorporated herein by reference.

In certain embodiments, the CPS technique may be used to detect and/or quantify a transient electrical signal generated by the movement of one or more charged species (i.e. ions) in solution. As the electrical signal of interest is transient, it only exists for a limited period of time. In many embodiments, this period of time generally ranges from about 1 minute to about 1 microsecond, usually from about 5 second to about 10 milliseconds. In addition, the transient electrical signal of interest is not a steady state signal, but is instead a signal that changes over time. In other words, the signal is a differential signal or parameter that is observed over a detection period. In various embodiments, the amplitude, length, shape and/or other characteristics of the signal may provide information about the underlying phenomenon being measured, such as a transient ionic flux.

In certain embodiments of the invention, depending on the type of detection unit utilized, the detected signal may represent a composite of all the ionic fluxes occurring simultaneously. For example, where the membrane-bounded vesicle is an intact cell, exposure to a regulatory agent or pharmaceutical agent may activate multiple ion channels simultaneously, such as potassium, sodium, calcium, chloride, phosphate, magnesium or other ion transporters. In such cases, it may be possible to deconvolute a composite signal by identifying the characteristics of the underlying ionic fluxes, which may differentially respond to the effects of various activators or inhibitors or may exhibit differential kinetic and/or regulatory properties. A variety of different detectable transitory electrical signals/parameters may be employed in the subject methods. Non-limiting examples of such signals include, but are not limited to: voltage, charge, current, impedance, etc. As indicated above, the signals that are measured are transitory and changing over the measurement period.

Generally, the transitory electrical signal or parameter of interest is detected by monitoring a sample medium for the transitory electrical signal. The sample medium is generally of a defined volume that may vary, but typically ranges from about 5 nanoliters to about 2 ml, usually from about 5 μl to about 0.1 ml and more usually from about 10 μl to about 0.05 ml.

The defined volume of medium may be monitored using any transitory electrical signal detection element. In many embodiments the detection element may comprise an electrode detection element, which includes at least one working electrode. The electrode(s) may be fabricated from any convenient material(s), including but not limited to metals, conductive polymers, carbon, silicon, polysilicon and the like. The electrode(s) can also be covered with thin isolators like glass, quartz, etc.

In many embodiments, the electrode detection element includes, in addition to the working electrode, a reference electrode. In certain embodiments, the electrode detection element includes a plurality of different electrodes. The electrodes may be configured in a variety of topologies, relative to each other, so long as they are capable of monitoring the sample in contact therewith for the transitory electrical signal generated by the ion fluxes.

In many embodiments, a noise reduction element may be employed, to remove the unwanted noise component from the detected transient electrical signal and provide a noise depleted signal. For example, a differential amplifier may be employed, e.g., a differential voltage or current amplifier, which receives input from the working and reference electrodes and provides a relatively or substantially noise depleted output signal.

In many embodiments, the devices or apparatuses employed to practice the subject methods are devices that include at least a first working electrode, a driver for the working electrode (and any other electrodes present on the device) (e.g., a differential amplifier to create a charge difference between the working and reference electrodes in a medium in contact therewith) and a signal processor for evaluating a response from the working electrode. In many embodiments, the device further includes at least one reference electrode and the signal processor evaluates the responses from both the working and reference electrode and compares them to generate the transitory electrical signal. In many embodiments, the device further includes a medium containment means in which the one or more electrodes may be contacted with a medium.

Devices

Also provided are devices for use in practicing the above described methods. The subject devices include at least: (1) an electrode detection element; and (2) an output signal processing element. Electrode detection elements may include at least one working electrode. In many embodiments, the electrode detection elements include two electrodes, one of which is a reference electrode. A sensor device may therefore include an output lead, working electrode and reference electrode. Transient electrical signals may be detected using the electrode sensor element and an output signal is generated and sent to an output signal processing element. A differential amplifier may be integrated into the sensor element. In certain embodiments, an array of such electrode sensing elements can also be employed, e.g., for use in the high throughput assaying of a number of individual samples, e.g., as may be found in the wells of a microtitre plate, where the array of electrode sensing elements may include a corresponding number of individual sensing elements.

In other embodiments, the electrode sensing element may be part of an integrated device that further includes a sample containment means. In many of these embodiments, the integrated device takes the form of a chip (e.g. “lab on a chip”) or array structure. A representative integrated sensor incorporating a CMOS chip may be used. The electrode array may be electrically connected via pads and/or solder balls (ball grid array packaging) to the CMOS chip. Sensors, differential amplifiers and signal routing may be embedded in the CMOS chip and the output signal may be available on a pad and/or solder ball. The electrode array itself can be placed on a quartz, glass, or silicon oxide covered planar wafer. To build the CMOS chip one can use any commercially available CMOS process as known to those of skill in the art.

In embodiments involving arrays of similar elements, each sensing element may be fluidically separated from other sensing elements on the array, e.g., by low walls or other types of fluid barriers (e.g., U.S. Pat. Nos. 5,807,522 and 5,545,531, the disclosures of which are herein incorporated by reference).

The subject devices may include a signal processing element. This signal processing element typically includes a software component and a hardware component, where the software component is made up of an appropriate algorithm recorded on a computer or processor readable storage medium. The algorithm present on the storage medium is one that reads the observed output transitory electrical signal provided by the electrode sensing element of the device and processes it to provide information about the underlying ion fluxes and/or transport processes. The computer or processor readable storage medium on which the algorithm is stored may be any convenient medium, including CD, DAT, floppy disk, RAM, ROM, etc, which medium is capable of being read by a hardware component of the device.

The above described integrated devices can take a variety of different formats, which formats include self-contained “lab on a chip” structures which include, in addition to the sample medium containment element and electrode sensing elements described above, various flow paths, junctions, etc., reagent ports, viewing windows, etc, all included in a microfluidic device. A multitude of different microfluidic devices are well known to those of skill in the art and may be readily modified to provide integrated devices of the present invention. Representative U.S. patents that describe various microfluidic devices and the structures present therein include, but are not limited to, U.S. Pat. Nos. 6,300,141; 6,287,850; 6,271,021; 6,251,343; 6,235,175; 6,213,151; 6,171,850; 6,123,819; 6,103,199; 6,054,277; and 5,976,336; the disclosures of which are incorporated herein by reference in their entirety.

Proteins

In different embodiments of the invention, transport and/or receptor proteins of interest may be assayed in their native state, for example in an intact cell, cell fragment or subcellular organelle. Alternatively, proteins of interest may be examined in a non-native state. For example, proteins may be: [1] purified from natural sources; [2] expressed by in vitro translation of an mRNA species or by linked transcription/translation of a DNA species; and/or [3] expressed in a host cell that has been transformed with a gene or a complementary DNA (cDNA) species. These methods are not limiting and proteins to be analyzed may be prepared by any method known in the art.

Protein Purification

In certain embodiments of the invention, proteins of interest may be partially or fully purified from a variety of sources before analysis. Protein purification techniques are well known in the art. These techniques typically involve an initial crude fractionation of cell or tissue homogenates and/or extracts into protein and non-protein fractions. Fractionation may utilize, for example, differential solubility in aqueous solutions, detergents and/or organic solvents, elimination of classes of contaminants such as nucleic acids by enzymatic digestion, precipitation of proteins with ammonium sulphate, polyethylene glycol, antibodies, heat denaturation and the like, followed by ultracentrifugation. Low molecular weight contaminants may be removed by dialysis, filtration and/or organic phase extraction.

Protein(s) of interest may be purified using chromatographic and/or electrophoretic techniques to achieve partial or complete purification. Methods suited to the purification of proteins, polypeptides and peptides include, but are not limited to, ion-exchange chromatography, gel exclusion chromatography, polyacrylamide gel electrophoresis, affinity chromatography, immunoaffinity chromatography, hydroxylapatite chromatography, hydrophobic interaction chromatography, reverse phase chromatography, isoelectric focusing, fast protein liquid chromatography (FPLC) and high pressure liquid chromatography (HPLC). These and other methods of protein purification are known in the art and are not limiting for the claimed subject matter. Any known method of protein purification may be used. There is no requirement that the protein must be in its most purified state. Methods exhibiting a lower degree of relative purification may, for example, have advantages in increased recovery of protein.

Particular embodiments of the invention may rely on affinity chromatography for purification of proteins. The method relies on an affinity between a protein and a molecule to which it can specifically bind. Chromatography material may be prepared by covalently attaching a protein-binding ligand, such as an antibody, antibody fragment, receptor protein, substrate, inhibitor, product or an analog of such ligands to an insoluble matrix, such as column chromatography beads or a nylon or other membrane. The matrix is then able to specifically adsorb the target protein from a solution. Elution occurs by changing the solvent conditions (e.g. pH, ionic strength, temperature, detergent concentration, etc.). One of the most common forms of affinity chromatography is immunoaffinity chromatography. Methods for generating antibodies against various types of proteins for use in immunoaffinity chromatography are well known in the art.

In some embodiments of the invention, one or more proteins of interest may be specifically labeled in order to facilitate purification. The protein of interest may be followed through a purification protocol by looking for the presence of labeled protein . In other embodiments of the invention, proteins may be post-translationally labeled using side chain specific and/or selective reagents as discussed below. Various methods for protein labeling are known in the art.

In Vitro Translation

Proteins may be expressed using an in vitro translation system with mRNA templates. Complete kits for performing in vitro translation are available from commercial sources, such as Ambion (Austin, Tex.), Promega (Madison, Wis.), Amersham Pharmacia Biotech (Piscataway, N.J.), Invitrogen (Carlsbad, Calif.) and Novagen (Madison, Wis.). Such kits may utilize total RNA, purified polyadenylated mRNA, and/or purified individual mRNA species obtained from a cell, tissue or other sample. Methods of preparing different RNA fractions and/or individual mRNA species for use in in vitro translation are known. (E.g., Sambrook, et al., Molecular Cloning: A Laboratory Manual, Cold Spring Harbor Laboratory Press, Cold Spring Harbor, N.Y., 1989; Ausubel et al., Current Protocols in Molecular Biology, Wiley and Sons, New York, N.Y., 1994).

Commonly used in vitro translation systems are based on rabbit reticulocyte lysates, wheat germ extracts and E. coli extracts. In vitro translation systems based on rabbit reticulocyte lysates are particularly robust and efficient for eukaryotic translation. The systems contain crude cell extracts including ribosomal subunits, transfer RNAs (tRNAs), aminoacyl-tRNA synthetases, initiation, elongation and termination factors and/or all other components required for translation. Other components of use in supplementing in vitro translation systems and methods of use of such systems are known in the art (e.g., www.ambion.com/basics/translation/translation101.html).

In certain alternative embodiments of the invention, in vitro translation may be linked to transcription of genes to generate mRNAs. Such linked transcription/translation systems may use PCR® amplification products and/or DNA sequences inserted into standard expression vectors such as BACs (bacterial artificial chromosomes), YACs (yeast artificial chromosomes), cosmids, plasmids, phage and/or other known expression vectors. Linked transcription/translation systems are available from commercial sources (e.g., Proteinscript™ II kit, Ambion, Austin, Tex.; Quick Coupled System, Promega, Madison, Wis.; Expressway, Invitrogen, Carlsbad, Calif.). Such systems may incorporate various elements to optimize the efficiency of transcription and translation, such as polyadenylation sequences, consensus ribosomal binding (Kozak) sequences, Shine-Dalgarno sequences and/or other regulatory sequences known in the art.

In different embodiments of the invention, expressed proteins may be purified from the crude in vitro translation mixture prior to analysis or alternatively may be analyzed without purification. The use of protein purification may depend in part on whether a crude RNA fraction or a purified RNA species is used as the template for translation.

Protein Expression in Host Cells

Nucleic acids encoding proteins of interest may be incorporated into expression vectors for transformation into host cells and production of the encoded proteins . Non-limiting examples of host cell lines known in the art include bacteria such as E. coli, yeast such as Pichia pastoris, and mammalian cell lines such as VERO cells, HeLa cells, Chinese hamster ovary cell lines, human embryonic kidney (HEK) 293 cells, mouse neuroblastoma N2A cells, or the W138, BHK, COS-1, COS-7, 293, HepG2, 3T3, RIN, L-929 and MDCK cell lines. These and other host cell lines may be obtained from standard sources, such as the American Type Culture Collection (Rockville, M.d.) or commercial vendors.

A complete gene can be expressed or fragments of a gene encoding portions of a protein can be expressed. The gene or gene fragment encoding protein(s) of interest may be inserted into an expression vector by standard cloning techniques. Expression libraries containing part or all of the messenger RNAs expressed in a given cell or tissue type may be prepared by known techniques. Such libraries may be screened for clones encoding particular proteins of interest, for example using antibody or oligonucleotide probes and known screening techniques.

The engineering of DNA segment(s) for expression in a prokaryotic or eukaryotic system may be performed by techniques generally known in the art. Any known expression system may be employed for protein expression. Expression vectors may comprise various known regulatory elements for protein expression, such as promoters, enhancers, ribosome binding sites, termination sequences, polyadenylation sites, etc.

Promoters commonly used in bacterial expression vectors include the P-lactamase, lactose and tryptophan promoter systems. Suitable promoter sequences in yeast expression vectors include the promoters for 3-phosphoglycerate kinase or other glycolytic enzymes. Promoters of use for mammalian cell expression may be derived from the genome of mammalian cells (e.g., metallothionein promoter) or from mammalian viruses (e.g., the adenovirus late promoter or the early and late promoters of SV40). Many other promoters are known and may be used in the practice of the disclosed methods.

Eukaryotic expression systems of use include, but are not limited to, insect cell systems infected with, for example, recombinant baculovirus, or plant cell systems infected with recombinant cauliflower mosaic virus or tobacco mosaic virus. In an exemplary insect cell system, Autographa californica nuclear polyhidrosis virus is used as a vector to express foreign genes in Spodoptera frugiperda cells or the Hi5 cell line (Invitrogen, Carlsbad, Calif.). Nucleic acid coding sequences are cloned into, for example, the polyhedrin gene of the virus under control of the polyhedrin promoter. Recombinant viruses containing the cloned gene are then used to infect Spodoptera frugiperda cells and the inserted gene is expressed (e.g., U.S. Pat. No. 4,215,051; Kitts et al., Biotechniques 14:810-817, 1993; Lucklow et al., J. Virol., 67:4566-79, 1993). Other exemplary insect cell expression vectors are based on baculovirus vectors, for example, pBlueBac (Invitrogen, Sorrento, Calif.).

An exemplary expression system in mammalian cell lines may utilize adenovirus as an expression vector. Coding sequences may be ligated to, e.g., the adenovirus late promoter. The cloned gene may be inserted into the adenovirus genome by in vitro or in vivo recombination. Insertion in a non-essential region of the viral genome (e.g., region E1 or E3) results in a recombinant virus that is capable of infecting and expressing cloned proteins in mammalian host cells. The disclosed examples are not limiting and any known expression vector may be used.

In certain embodiments of the invention, cells transformed with expression vectors may be selected from non-transformed cells. A number of selection systems may be used, including but not limited to, the thymidine kinase gene, hypoxanthine-guanine phosphoribosyltransferase gene, methotrexate resistance gene, neomycin phosphotransferase gene and hygromycin resistance gene. These genes, contained in standard cloning vectors, either confer resistance to cytotoxic agents or allow cell growth in nutrient deficient medium.

Expressed proteins may be partially or completely purified before analysis. In some embodiments of the invention, protein purification may be facilitated by expressing cloned sequences as fusion proteins containing short leader sequences that allow rapid affinity purification. Examples of such fusion protein expression systems are the glutathione S-transferase system (Pharmacia, Piscataway, N.J.), the maltose binding protein system (NEB, Beverley, Mass.), the FLAG system (IBI, New Haven, Conn.), and the 6xHis system (Qiagen, Chatsworth, Calif.). In one embodiment of the invention, the leader sequence is linked to a protein by a specific recognition site for a protease. Examples of suitable sequences include those recognized by the Tobacco Etch Virus protease (Life Technologies, Gaithersburg, M.d.) or Factor Xa (New England Biolabs, Beverley, Mass.). Alternatively, expressed proteins may be purified by standard techniques discussed above.

Lipid Vesicles

Liposomes

In certain embodiments of the invention, the ion transporters and/or cell receptor proteins of interest may be obtained in a native membrane-bound vesicle, such as an intact cell, cell fragment, mitochondrion, chloroplast or other subcellular organelle. In alternative embodiments, proteins of interest may be partially or fully purified and incorporated into a synthetic lipid vesicle, such as a liposome.

Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. (Ghosh and Bachhawat, In: Liver Diseases, Targeted Diagnosis and Therapy Using Specific Receptors and Ligands. Wu et aL, eds., Marcel Dekker, New York, pp. 87-104, 1991.) In certain embodiments, unilamellar liposomes may be used to provide a simpler transport system. Unilamellar liposomes may be formed by brief sonication of solutions comprising multilamellar liposomes, by methods well known in the art.

Cross-linkers

Bifunctional cross-linking reagents have been extensively used for a variety of purposes including preparation of affinity matrices, modification and stabilization of diverse structures, identification of ligand and receptor binding sites, and structural studies. Homobifunctional reagents that carry two identical functional groups proved to be highly efficient in inducing cross-linking between identical and different macromolecules or subunits of a macromolecule, and linking of polypeptide ligands to their specific binding sites. Heterobifimctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, sulfhydryl, guanidino, indole, carboxyl specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

Exemplary methods for cross-linking ligands to liposomes are described in U.S. Pat. No. 5,603,872 and U.S. Pat. No. 5,401,511, each specifically incorporated herein by reference in its entirety). Various ligands can be covalently bound to liposomal surfaces through the cross-linking of amine residues. Liposomes, in particular, multilamellar vesicles (MLV) or unilamellar vesicles such as microemulsified liposomes (MEL) and large unilamellar liposomes (LUVET), each containing phosphatidylethanolamine (PE), have been prepared by established procedures. The inclusion of PE in the liposome provides an active functional residue, a primary amine, on the liposomal surface for cross-linking purposes. Ligands such as epidermal growth factor (EGF) have been successfully linked with PE-liposomes. Ligands are bound covalently to discrete sites on the liposome surfaces. The number and surface density of these sites are dictated by the liposome formulation and the liposome type. The liposomal surfaces may also have sites for non-covalent association. To form covalent conjugates of ligands and liposomes, cross-linking reagents have been studied for effectiveness and biocompatibility. Cross-linking reagents include glutaraldehyde (GAD), bifunctional oxirane (OXR), ethylene glycol diglycidyl ether (EGDE), and a water soluble carbodiimide, preferably 1-ethyl-3-(3-dimethylaminopropyl) carbodiimide (EDC). Through the complex chemistry of cross-linking, linkage of the amine residues of the recognizing substance and liposomes is established.

In another example, heterobifunctional cross-linking reagents and methods of using the cross-linking reagents are described (U.S. Pat. No. 5,889,155, incorporated herein by reference in its entirety). The cross-linking reagents combine a nucleophilic hydrazide residue with an electrophilic maleimide residue, allowing coupling in one example of aldehydes to free thiols. The cross-linking reagent can be modified to cross-link various functional groups.

### EXAMPLES

### Example 1

Detection of Ion Fluxes by Charge Perturbation Signature

Membrane Bound Vesicles

In an exemplary embodiment of the invention, a lipid vesicle (or any type of biological or synthetic vesicle) is made containing a protein of interest. The protein can be any cell surface receptor (e.g., a proton pump) and/or transport protein. Where channel forming proteins are to be analyzed, the channel can be any type of channel protein (i.e., ligand-gated, mechanically-gated, voltage-gated, light-gated).

Once the vesicle is made it may be placed in proximity to an electrode for charge perturbation signature (CPS) analysis. A linking agent might be used to spatially confine the vesicle, or the vesicle may be immobilized by placing it into a gel matrix in proximity to an electrode. A variety of cross-linking agents and methods are well known in the art and any such known methods and agents may be used. For example, lipid vesicles may be synthesized by standard techniques, incorporating biotinylated lipids (e.g., biotin-longchain-dipalmitoyl phosphatidylethanolamine (biotin-LC-DPPE) from Pierce Chemical Ltd., Rockford, Ill.). Gold coated electrodes, either custom made or commercially obtained (e.g., BTX Products, Holliston, Ma.), may be coated with streptavidin by incubation in a low ionic strength Tris-HCl buffer solution. The streptavidin is naturally sticky and attaches to the gold without any further modification. Alternatively, streptavidin or avidin attachment to gold electrode surfaces may be enhanced by covalent modification, for example with sulfhydryl groups, which form covalent linkages to gold surfaces. Attachment is facilitated by cleaning the gold surface, for example with dilute acid, before incubation with streptavidin. The biotinylated lipid vesicles attach to the streptavidin moieties located on the electrode surface, thus localizing the vesicles in the proximity of the electrode.

In various embodiments, a bioarray of vesicles, each of which may contain different receptor proteins (potentially of the same family, i.e the various subfamilies of NMDA receptors) is exposed to an external perturbation to activate ion channels or a transmembrane ion flux. The external perturbation can be mechanical, chemical, optical, or ionic gradients.

In an examplary application in drug discovery, a specific drug compound to be tested is added to the medium. The drug may bind to one or more specific ligand-gated channels to open the channel. The resulting ion flux may be monitored by CPS assay. In the case where different medium containing compartments (e.g., different wells of a microtiter well format) contain vesicles, each of which incorporates and different receptor and/or transport protein, the presence or absence of an ionic flux in that compartment represents the presence or absence of a protein that can be activated or inhibited by the drug of interest. In such a way, it is possible to assay a large number of transport and/or receptor proteins for binding to a particular pharmaceutical agent or to screen a library of compounds for activity.

The skilled artisan will realize that in various formats, it is possible to assay for inhibition or activation of a receptor protein that may itself not exhibit ion transport properties. For example, a receptor protein may interact with another membrane protein, such as a channel forming protein or active transport protein. Binding of a drug, activator or inhibitor to the receptor protein may in turn act to open or close the linked channel protein or active transport protein. In certain embodiments, it is possible to form vesicles where a variety of receptor proteins may activate or inhibit the same transport protein.

CPS Assay

Using a bilayer enclosed vesicle with one or more receptor proteins, a temporal CPS signal resulting from a diffusion potential may be measured while the influx or efflux of ions occurs across the membrane. The measured voltage change is correlated to the quality and quantity of the membrane channels that are activated or inhibited.

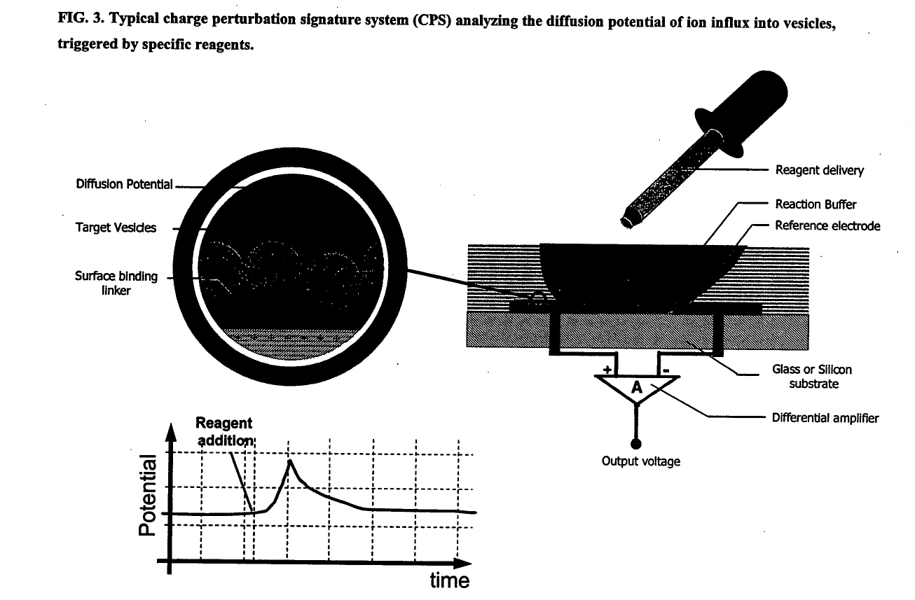

An exemplary CPS system is illustrated in FIG. 3. The differential potential between a reference electrode and an active electrode, associated with vesicles containing target molecules, is measured during the ion influx into the vesicle, after addition of one or more triggering reagents. The reagents may include, without limitation, drugs, pharmaceutical agents, extracts of plants, animals or microbes, hormones, neurotransmitters, toxins, poisons, steroids or any other compound that may activate or inhibit an ion transport process. In certain embodiments, the addition of a first compound known to activate an ion transporter may be followed by the addition of a variety of second compounds that may potentially inhibit the same transport process. Alternatively, an inhibitor may be added first to block the effect of known activators.

The membrane bound vesicle array disclosed herein exhibits certain advantages in applications involving drug discovery. These include, but are not limited to: (1) the ability to display natural cell membrane behavior in an in vitro format; and (2) the ability to assay membrane components in array format. The disclosed methods are also of use to screen putative antimicrobial agents by detecting ionic flux resulting from the lysis of cell membranes.

All of the COMPOSITIONS, METHODS and APPARATUS disclosed and claimed herein can be made and executed without undue experimentation in light of the present disclosure. While the compositions and methods of this invention have been described in terms of preferred embodiments, it will be apparent to those of skill in the art that variations may be applied to the COMPOSITIONS, METHODS and APPARATUS and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

References

The following references, to the extent that they provide exemplary procedural or other details supplementary to those set forth herein, are specifically incorporated herein by reference.

Groves J T, and Boxer S G., “Micropattem formation in supported lipid membranes,” Acc Chem Res 2002 Mar;35(3):149-57

U.S. patent application: 20020164777

U.S. patent application: 20020064841

U.S. patent application: 20020108869

U.S. patent application: 20020009807

What is claimed is:

1. A method comprising:
a) obtaining a membrane bound vesicle comprising at least one ion transporter;
b) adding an agent to affect ion flux through the transporter;
c) generating a transient electrical signal from the ion flux; and
d) using charge perturbation signature to measure the transient electrical signal.

2. The method of claim 1, further comprising placing the vesicle in proximity to a working electrode.

3. The method of claim 2, further comprising using a reference electrode to reduce background noise.

4. The method of claim 1, wherein the agent is a ligand, voltage, light or mechanical agent.

5. The method of claim 5, wherein the ligand is a drug, pharmaceutical agent, plant extract, animal extract, microbial extract, hormone, cytokine, chemokine, neurotransmitter, toxin, poison or steroid.

6. The method of claim 1, wherein the vesicle further comprises a receptor protein associated with the ion transporter.

7. The method of claim 6, wherein the receptor protein activates or inhibits the ion transporter.

8. The method of claim 7, wherein the agent activates or inhibits the receptor protein.

9. The method of claim 1, wherein the ion is selected from the group consisting of hydrogen, potassium, sodium, calcium, magnesium, chloride, phosphate, sulfate and nitrate ions.

10. The method of claim 2, wherein the working electrode is a pH electrode.

11. The method of claim 2, wherein the working electrode detects voltage, current or resistance.

12. The method of claim 2, wherein the working electrode is an ion-selective electrode.,

13. The method of claim 2, wherein an array of electrodes is associated with a corresponding array of fluid filled compartments in a multiplex format.
